(19) United States
(12) Patent Application Publication
(10) Pub. No.: US 2021/0322196 A1
(43) Pub. Date: Oct. 21, 2021

(54) ORTHOSIS OR EXOSKELETON SYSTEM

(71) Applicant: OPUM Technologies Limited, Auckland (NZ)

(72) Inventor: Andrew John McDaid, Hamilton (NZ)

(21) Appl. No.: 17/271,954

(22) PCT Filed: Aug. 28, 2019
(86) PCT No.: PCT/NZ2019/050110

§ 371 Date: Feb. 26, 2021

(30) Foreign Application Priority Data
Aug. 28, 2018 (NZ) 745117

(51) Int. Cl. A61F 5/01; A61H 3/00

(52) U.S. Cl. CPC A61F 5/0102; A61H 3/00; A61F 5/0123; A61H 2003/007; A61H 2201/5064; A61H 2201/5079; A61F 2005/0167; A61F 2005/0179; A61H 2201/165; A61H 2201/1676; A61H 2201/5069; A61F 2005/0137

(57) ABSTRACT

An orthosis or exoskeleton system can be mounted proximate a user's joint. The system includes a brace, which includes a first body mounting portion to be mounted to one side of the user's joint and a second body mounting portion to be mounted to an opposite side of the user's joint. The first and second mounting portions are moveably coupled together. A range of motion (ROM) limitation mechanism presents a number of selectable ROM limits between the first and second body mounting portions. One or more sensors detect a user's kinematics and/or kinetics. The system is configurable during use based on the user's kinematics and/or kinetics, including by setting or updating the ROM limitation mechanism from a present ROM limit setting to a new ROM limit setting.

### FIELD OF INVENTION

The present invention relates to a patient attachment system such as an orthosis, exoskeleton or rehabilitation robot for use in the rehabilitation, support, monitoring, diagnosis or prevention of afflictions and/or injuries of a human or animal patient, neuromuscular, skeletal and/or otherwise.

### BACKGROUND TO THE INVENTION

Individuals suffering from injury and disease commonly require a range of rehabilitative strategies with the objective of recovering to maximise their ability and reducing time to reach that maximum ability. Individuals may additionally or separately need assistance to compensate for any lost function. Additionally, healthy individuals may wish to improve their abilities beyond their current capability and or enhance function.

Robotic and sensorised devices such as orthosis systems (orthotics) or exoskeletons may be useful in a number of fields including rehabilitation, assessment, assistance, performance improvement, but show particular promise in the field of rehabilitation, for example for ACL injuries or stroke victims.

At an early (acute) impairment stage a human has a different ability and hence different requirements than in the later (chronic) stages.

A patient may typically have different rehabilitative needs at different stages of injury and recovery, which require different systems and methods of treatment at various times. A patient may typically require varying therapy and assistance plans throughout the recovery process.

A number of problems or challenges exist with the systems and methods of rehabilitation of the prior art.

One problem is that to achieve maximum benefit to a patient or user, a configuration of an orthosis/exoskeleton, or a type of orthosis/exoskeleton, needs to be adjusted or needs to be changed as the patient or user develops or progresses through their plan (for example, from injury to recovery). Adjustment or adaption of an orthosis or exoskeleton is primarily done on a subjective manner by the user or an expert.

Adjustment of an orthosis/exoskeleton may not be carried out at an optimum time, for example adjustment may be made on a periodic basis when a patient attends a consultation with a medical expert, rather than when adjustment would have ideally been made to achieve maximum benefit to the patient. Attending consultations is time consuming and can be expensive, and progress to recovery may be less effective than if more frequent configuration adjustments had been made over time.

Another problem is that the wearable apparatuses can expensive and complex, especially apparatuses requiring sensors providing data.

A further challenge is the need for an efficient system for or method of delivery of rehabilitation in remote and/or rural settings where systems are necessarily prescribed and/or delivered to patients to affect rehabilitation without the need of a trained clinician to physically visit the patient to update the system.

### OBJECT OF THE INVENTION

It is an object of the invention to provide an orthosis or exoskeleton system and/or a method for configuring such a system that addresses one or more of the above-mentioned problems, or to at least provide the public with a useful choice.

### SUMMARY OF THE INVENTION

According to a first aspect of the invention, there is provided an orthosis or exoskeleton system comprising a brace comprising a first body mounting portion to be mounted to one side of the user's joint and a second body mounting portion to be mounted to an opposite side of the user's joint, the first and second mounting portions moveably coupled together,

a range of motion (ROM) limitation mechanism presenting a plurality of selectable ROM limits between the first and second body mounting portions,

one or more sensors to detect a user's kinematics and/or kinetics, and wherein the system is configurable during use based on the user's kinematics and/or kinetics, including by setting or updating the ROM limitation mechanism from a present ROM limit setting to a new ROM limit setting.

In a preferred embodiment, the system detects a user's kinematics and/or kinetics through relative movement of the first and second mounting portions.

The System Configuration is Determined by/Based on:

In a preferred embodiment, the one or more sensors detect at least a user's range of motion (ROM) between the first and second mounting portions, and the system is configurable based on the user's ROM being greater than a predetermined ROM threshold. For example, the user's ROM being greater than a predetermined ROM threshold is determined by any one or more of:
an average of the user's ROM being greater than a predetermined threshold,
the user's ROM being greater than a predetermined ROM threshold more than a predetermined number of times,
the user's ROM being greater than a predetermined ROM threshold more than a predetermined number of times within a predetermined time period.

The predetermined ROM threshold may be an angle of displacement between the first and second mounting portions and/or a resistance to motion threshold between the first and second mounting portions.

In some embodiments, the system is configurable based on the user reaching an ROM limit.

in some embodiments, the sensors detect at least one of a ROM limit being reached and a resistance to movement (RTM) between the first and second mounting portions, and the system is configurable based on at least one of the user reaching an ROM limit and a RTM threshold achieved by the user. The system may be configured based on any one or more of:
the user hitting a ROM limit and/or achieving a RTM threshold more than a predetermined number of times,
the user hitting a ROM limit and/or achieving a RTM threshold more than a predetermined number of times within a predetermined time period,
an average of the user's RTM being greater than a predetermined threshold.

The predetermined RTM threshold may be a torque or force applied by one of or between the first and second mounting portions and/or a ROM threshold or limit between the first and second mounting portions.

The system may be configurable based on other indicators of the user's kinematics. For example, in some embodiments, the one or more sensors detect at least one of the user's steps and a user's ground step force, and the system is configurable based on a number of steps taken by a user being greater than a predetermined threshold and/or the user's ground reaction force being greater than a force threshold. The system may have a user interface to receive a user pain score or other physiological factor, to be entered by the user, and the system configurable based on the user entered pain score/physiology factor. The system may be configurable based on historical data for a population of previous patients with similar information/demographics to the user, the historical data including one or more of gender, age and injury type, and time since injury.

System Configurations, e.g. Setting Stop, Setting Resistance to Motion Level, Selection of a Body Mounting Portion, Setting ROM Threshold, Setting RTM Threshold, Including Manual or Automatic

The system may be configurable in a number of different ways. In some embodiments, the system a resistance to movement (RTM) mechanism presenting a plurality of RTM levels between the first and second mounting portions, and the system is configurable by setting a said RTM level, i.e. setting or updating the RTM mechanism from a present RTM level to a new RTM level. For example, the system sets a limit or level by incrementing or decrementing a ROM limit and/or a RTM level from a present limit or level to a next limit or level. The system may increment or decrement a ROM limit or RTM level from a present limit or level to a next limit or level if the next limit or level is less than a maximum or greater than a minimum limit or level. If the present ROM limit is a maximum limit the system maybe further configured by removing the ROM limit so that system no longer provides a ROM limit.

The ROM limitation mechanism comprises a plurality of mechanical stops. The system is configured by selection of one of the plurality of mechanical stops to set the ROM limit, and/or

wherein the RTM mechanism comprises a plurality of resilient members, each providing a RTM level, and wherein the system is configurable by selection of one of the plurality of resilient members to set the RTM level, or

wherein the RTM mechanism comprises a resilient member with a plurality of adjustable levels of tension or compression, and wherein the system is configurable by selection of one of the plurality of levels of tension or compression to set the RTM level.

In some embodiments, the system comprises a plurality of braces and/or a plurality of first body mounting portions and a plurality of second body mounting portions, each of the braces and/or the first and second mounting portions having different sizes and/or types and/or configurations with respect to each other, and wherein the system is configurable by selecting a said brace and/or selectively coupling together a said first and second mounting portions. The system may be configured by updating the brace from a rigid or semi-rigid brace to a compression sleeve brace.

In some embodiments, the system comprises at least one actuator to automatically set the ROM limit or the RTM level based on the user's kinematics. The system may be configurable during use by setting or updating an ROM threshold and/or a RTM threshold from a present threshold to a new threshold. The new ROM threshold may be equal to the new ROM limit minus an offset. The system may comprise a user interface and/or communication device to communicate to the user or a user device that a change to the ROM/RTM threshold, ROM limit, the RTM level or the size and/or configuration is required. The user can then make the necessary change, without a requirement to consult a medical expert.

Detecting ROM/RTM:

The one or more sensors may detect a ROM and/or RTM by one or more of:
measuring displacement of the first and/or second mounting portions,
measuring relative (angular) displacement between the first and second mounting portions,
detecting an ROM limit has been reached,
measuring a torque applied by the first and/or second mounting portions,
measuring a force applied by the first and/or second mounting portions.

Detecting RTM:

In some embodiments the RTM mechanism comprises at least one resilient member to present resistance to movement between the first and second mounting portions. The one or more sensors detects a RTM by measuring an elastic deformation or displacement of the resilient member.

Detecting ROM Limit Contact:

Based on User's Kinematics

In some embodiments and in a further aspect of the present invention, the system determines a ROM limit has been reached based on the user's kinematics. For example, the system determines a ROM limit has been reached based on one or more derivatives of a relative displacement (position) and/or a rate of change of RTM between the first and second mounting portions. The system may determine a ROM limit has been reached based a rate of change in relative displacement and/or velocity between the first and second mounting portions. In some embodiments, the system determines a ROM limit has been reached based on a change in velocity between the first and second mounting portions from non-zero velocity to zero velocity, and/or when the velocity between the first and second mounting portions is zero for a predetermined time period, and wherein the ROM limit is defined by an angle of displacement between the first and second body mounting portions when the velocity between the first and second mounting portions is zero or zero for the predetermined time period.

Based on Detecting Contact

The one or more sensors may comprise an electrical system, and the ROM limit being reached is detected by a change in state of the electrical system. The change in state is one or more of a change in resistance, capacitance, inductance, a position of an electrical switch. The electrical system may comprise a first electrical contact associated with the ROM limit and a second electrical contact associated with the first or second mounting portion, and the ROM limit being reached is detected when the first and second electrical contacts connect. In another example, an electrical switch may be activated by contact between the limit and the first and/or second mounting portion.

Determining a ROM Limit or RTM Level Setting

In some embodiments, the system comprises a range of movement (ROM) limitation mechanism presenting a plurality of ROM limits and/or a resistance to movement (RTM) limitation mechanism presenting a plurality of RTM levels between the first and second mounting portions, and one or more sensors to determine a present ROM limit setting, and/or
a present RTM level setting. This can be used as feedback to the system or the user of the present setting for the ROM limit or RTM level. For example, wherein the system includes a actuator to automatically configure the system from a present ROM to a wider or narrower ROM, the system can increment or decrement the limit position as necessary, until a maximum or minimum is position is reached, as indicated by the sensed actual position of the limit.

The one or more sensors may comprise an electrical system to determine the present ROM limit setting and/or the present RTM setting, by any one or more of resistance, capacitance, inductance, a position of an electrical switch.

Type of Sensors

The one or more sensors comprise one or more of:
a rotary encoder(s) to measure an angle of displacement between the first and second mounting portions,
a multi-dimensional sensor to measure all 3 angular motions of the joint (e.g. a sensorised linkage mechanism)
an inertial measuring unit (may include accelerometers, gyroscopes, magnetometers) attached to one or each of the first and second mounting portions to measure an angle of displacement between the first and second mounting portions,
an electrical circuit or switch to detect when a ROM limit has been reached,
a strain gauge to measure elastic deformation or displacement of a resilient member elastically deformed by relative movement between the first and second mounting portions to detect a RTM,
an accelerometer to measure a rate of change in velocity of the first and/or second mounting portion to detect when the ROM limit has been reached,
a gyroscope to measure angular velocity of the first and/or second mounting portion.

The system comprises a processor or a communication device to communicate data to a processor. For example, the processor may be comprised in a user's smartphone. The processor is configured to determine one or more of a user's kinematics, a present position of a range of motion limit, a contact with a range of motion limit, a resistance to motion achieved by a user, and a resistance to motion level and/or to provide an output to cause any one or more of a ROM threshold, a RTM threshold, a ROM limit and/or a RTM level to be automatically set or updated.

According to a second aspect of the invention, there is provided an orthosis or exoskeleton system to be mounted proximate a user's joint, the system comprising:
a brace comprising a first body mounting portion to be mounted to one side of the user's joint and a second body mounting portion to be mounted to an opposite side of the user's joint, the first and second mounting portions moveably coupled together,
one or more sensors to detect a user's kinematics and/or kinetics, and
wherein the system is configurable during use based on the user achieving a predetermined kinematic and/or kinetic threshold, and
wherein the system is configured automatically during use by automatically updating the kinematic and/or kinetic threshold from a present threshold to a new threshold.

According to a third aspect of the invention, there is provided an orthosis or exoskeleton system comprising a brace comprising a first body mounting portion to be mounted to one side of the user's joint and a second body mounting portion to be mounted to an opposite side of the user's joint. The first and second mounting portions are moveably coupled together. One or more sensors detect at least one of: a range of motion between the first and second mounting portions, a resistance to motion between the first and second mounting portions, and a range of motion limit being reached. The system is configurable during use based on at least one of (i) a range of motion (ROM) threshold achieved by a user, (ii) a resistance to motion (RTM) level achieved by the user, and the user reaching a ROM limit.

According to a fourth aspect of the invention, there is provided an orthosis or exoskeleton system comprising a brace comprising a first body mounting portion to be mounted to one side of the user's joint and a second body mounting portion to be mounted to an opposite side of the user's joint. The first and second mounting portions are moveably coupled together. A range of motion (ROM) limitation mechanism and/or a resistance to movement (RTM) mechanism present a ROM limit or level of RTM between the first and second mounting portions. One or more sensors detect a user's kinematics and/or kinetics through relative movement of the first and second mounting portions. The system determines a ROM limit has been reached based on the user's kinematics and/or kinetics.

According to a fifth aspect of the invention an orthosis or exoskeleton system comprises a brace comprising a first body mounting portion to be mounted to one side of the user's joint and a second body mounting portion to be mounted to an opposite side of the user's joint. The first and second mounting portions are moveably coupled together. A range of movement (ROM) limitation mechanism presents a plurality of ROM limits and/or a resistance to movement (RTM) limitation mechanism presents a plurality of RTM levels, between the first and second mounting portions. The one or more sensors to determine: a present ROM limit setting, and/or a present RTM level setting.

The second, third, fourth and fifth aspects may include any one or more of the features described above in relation to the first aspect of the invention.

Method of Configuring an Orthosis or Exoskeleton System

According to a fifth aspect of the invention, there is provided a method for configuring an orthosis or exoskeleton system comprising a brace, the brace having a first body mounting portion to be mounted to one side of the user's joint and a second body mounting portion to be mounted to an opposite side of the user's joint, the first and second mounting portions moveably coupled together, and a range of motion (ROM) limitation mechanism presenting a plurality of selectable ROM limits between the first and second body mounting portions, the method comprising:
measuring a user's kinematics and/or kinetics, and configuring the system during use based on the user's kinematics and/or kinetics, including configuring the system by setting or updating the ROM limitation mechanism from a present ROM limit setting to a new ROM limit setting.

In a preferred embodiment, the method includes detecting a user's kinematics and/or kinetics through relative movement of the first and second mounting portions.

In a preferred embodiment, the method comprises:
measuring at least a range of motion (ROM) between the first and second mounting portions, and
configuring the system based on the user's ROM being greater than a predetermined ROM threshold.

For example, the user's ROM being greater than a predetermined ROM threshold is determined by any one or more of:
an average of the user's ROM being greater than a predetermined threshold,
the user's ROM being greater than a predetermined ROM threshold more than a predetermined number of times,
the user's ROM being greater than a predetermined ROM threshold more than a predetermined number of times within a predetermined time period,
kinematic patterns during specific tasks e.g. gait, or an active ROM (AROM) exercise/assessment.

In some embodiments, the method comprises configuring the system based on the user reaching an ROM limit.

In some embodiments, the method comprises:
detecting a RTM between the first and second mounting portions, and
configuring the system based on the user reaching a RTM threshold.

The method may configure the system based on any one or more of:
the user hitting a ROM limit and/or achieving a RTM threshold more than a predetermined number of times,
the user hitting a ROM limit and/or achieving a RTM threshold more than a predetermined number of times within a predetermined time period,
an average of the user's RTM being greater than a predetermined threshold.

Additionally, the method may configure the system based on:
a number of steps taken by a user being greater than a predetermined threshold and/or
the user's ground reaction force being greater than a force threshold, and/or
a user psychological/pain score.

In some embodiments, the method comprising configuring the system based on historical data for a population of previous patients with similar information/demographics to the user, the historical data including one or more of gender, age and injury type, and time since injury

System Configurations, e.g. Setting Stop, Setting Resistance to Motion, Selection of a Brace, Including Manual or Automatic:

In a preferred embodiment, the system comprises a resistance to movement (RTM) mechanism presenting a plurality of RTM levels between the first and second mounting portions, and the method comprises configuring the system by setting or updating the RTM mechanism from a present RTM level to a new RTM level.

Preferably the method comprises setting the ROM limit or RTM level by incrementing or automatically decrementing a ROM limit and/or a RTM level from a present limit or level to a next limit or level in a plurality of limits/levels. Preferably the method comprises incrementing or decrementing a ROM limit or RTM level from a present limit or level to a next limit or level if the next limit or level is less than a maximum or greater than a minimum limit or level. IN some embodiments, the method comprises removing the ROM limit if the present ROM limit is a maximum limit so that the system no longer provides the user with a ROM limit.

In some embodiments, the system comprises a plurality of braces and/or a plurality of first body mounting portions and a plurality of second mounting portions, each of the braces and/or the first and second body mounting portions having different sizes and/or configurations with respect to each other, and wherein the method comprises indicating to the user that a change in the brace and/or the first and/or second body mounting portions is required. In some embodiments, the method comprises instructing the user to update the brace from a rigid or semi-rigid brace to a compression sleeve brace.

In some embodiments, the method comprises automatically configuring the system by automatically setting the ROM limit and/or the RTM level based on the user's kinematics and/or kinetics. In some embodiments, the method comprises configuring the system during use by setting or updating an ROM threshold and/or a RTM threshold from a present threshold to a new threshold. In some embodiments, the new ROM threshold is equal to the new ROM limit minus a ROM offset. In some embodiments, the system comprises a user interface and/or communication device to communicate to the user or a user device, and the method comprises communicating to the user that a change to the ROM/RTM threshold, the ROM limit, the RTM level or the size and/or configuration is required. The user can then make the necessary change, without a requirement to consult a medical expert.

Detecting ROM/RTM:

In some embodiments, the method comprises detecting a ROM and/or a RTM by one or more of:
measuring displacement of the first and/or second mounting portions,
measuring relative (angular) displacement between the first and second mounting portions,
detecting an ROM limit has been reached,
measuring a torque applied by the first and/or second mounting portions,
measuring a force applied by the first and/or second mounting portion.

Detecting RTM:

In some embodiments, the RTM mechanism comprises at least one resilient member to present resistance to movement between the first and second mounting portions, and the method comprises detecting a RTM level by measuring an elastic deformation or displacement of the resilient member.

Detecting ROM Limit:

Based on User's Kinematics

In some embodiments, and in a further aspect of the invention, the method comprises determining an ROM limit has been reached based on the user's kinematics. The method may determine a ROM limit has been reached based on one or more derivatives of a relative displacement (position) and/or a rate of change of RTM between the first and second mounting portions. For example, the method determines a ROM limit has been reached based a rate of change in relative displacement and/or velocity between the first and second mounting portions. In some embodiments, the method comprising determining a ROM limit has been reached based on:
a change in velocity between the first and second mounting portions from non-zero velocity to zero velocity, and/or
when the velocity between the first and second mounting portions is zero for a predetermined time period, and wherein the ROM limit is defined by an angle of displacement between the first and second body mounting portions when the velocity between the first and second mounting portions is zero or zero for the predetermined time period

Determining a ROM Limit or RTM Level (Regardless of there being a Contact with a Limit)

In some embodiments, the system comprises a range of movement (ROM) limitation mechanism presenting a plurality of ROM limits and/or a resistance to movement (RTM) limitation mechanism presenting a plurality of RTM levels between the first and second mounting portions, and the method comprises determining:
a present ROM limit setting, and/or
a present RTM level setting.

In this specification and claims, unless the context suggests otherwise, the term ‘threshold’ means a setpoint/control point, i.e. a setpoint implemented in software, and the term ‘limit’ means a mechanical limit, such as a mechanical stop that physically ends or limits motion in a particular direction.

Further aspects of the invention, which should be considered in all its novel aspects, will become apparent to those skilled in the art upon reading of the following description which provides at least one example of a practical application of the invention.

### BRIEF DESCRIPTION OF THE DRAWINGS

One or more embodiments of the invention will be described below by way of example only, and without intending to be limiting, with reference to the following drawings, in which:

FIG. 1A is a front view of an assembled orthosis system according to an embodiment of the present invention.

FIG. 2 is a front view of an assembled orthosis system according to another embodiment of the present invention.

FIG. 3 is a schematic representation of an orthosis system according to an embodiment of the present invention.

FIG. 3B is a schematic representation of a sensor electrical circuit.

FIG. 4 is a schematic representation of an orthosis system according to another embodiment of the present invention.

FIG. 5 a schematic representation of an orthosis system according to another embodiment of the present invention.

FIG. 6 a schematic representation of an orthosis system according to another embodiment of the present invention.

FIG. 7 is a flow chart of a method for configuring an orthosis system according to another embodiment or aspect of the present invention.

FIG. 8 is a flow chart of a method for configuring an orthosis system according to another embodiment or aspect of the present invention.

FIG. 9 is a flow chart of a method for configuring an orthosis system according to another embodiment or aspect of the present invention.

FIG. 10 is a flow chart of a method for configuring an orthosis system according to another embodiment or aspect of the present invention.

FIG. 11 is a flow chart of a method for configuring an orthosis system according to another embodiment or aspect of the present invention.

FIG. 12 is a flow chart of a method for configuring an orthosis system according to another embodiment or aspect of the present invention.

FIG. 13 is a schematic chart of a user's joint angle α (e.g. an angle between the brace assemblies) and the velocity v of the user's limb.

### BRIEF DESCRIPTION OF PREFERRED EMBODIMENTS OF THE INVENTION

It will be understood that embodiments of the orthosis system herein disclosed may be configured as a brace for any part of a patient's body. While the preferred embodiments relate to a knee brace and an elbow brace, the orthosis system of the present invention may alternatively be configured as, for example, a wrist, elbow, shoulder or ankle brace with suitable adaptations to the shape and size of the components of the orthosis system. The brace may also have more than one degree of motion, e.g. could consist of 3 rotations and/or three translations. The patient may be a human or animal patient.

Embodiments of the invention will be described comprising rigid frame elements providing structural support to the orthosis system or brace and softer and/or less rigid pad elements providing comfort to those parts of the system in contact with the patient. In alternative embodiments the body attachment module comprises a fabric-based brace or wearable garment to be worn under clothing or strapping to remain more lightweight, for example for wearing throughout the day when less injured.

Knee Brace

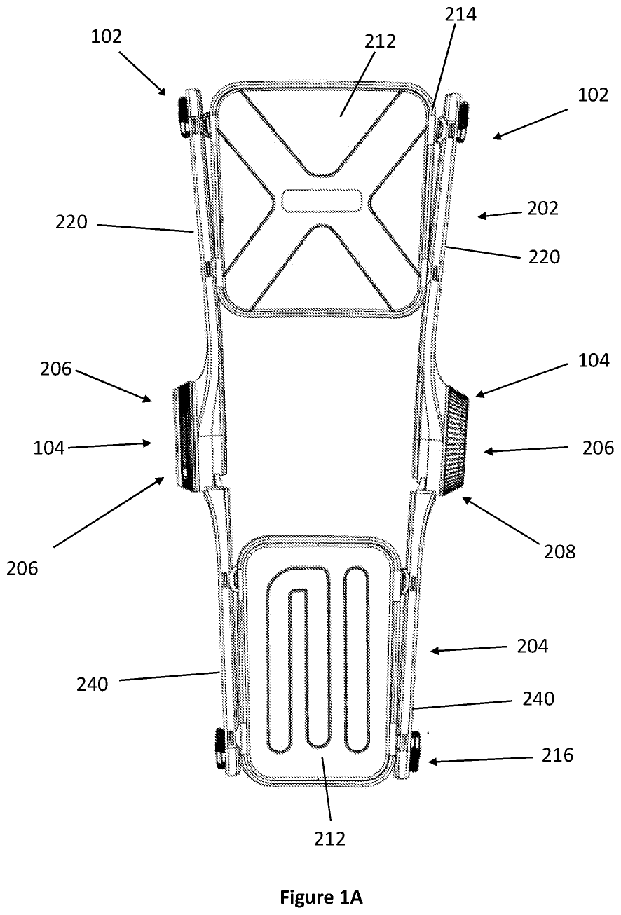

FIGS. 1A and 1B show an example orthosis system 200, as described in PCT application PCT/NZ2018/050085, the contents of which are incorporated herein by reference. Orthosis system 200 is particularly suited for mounting proximate a knee (not shown) of the patient (not shown).

The system 200 has a brace or body mounting module 102 comprising a first body mounting portion 202 and a second body mounting portion 204. In use, the first body mounting portion 202 is mounted upwardly of the knee of the patient and the second body mounting portion 204 is mounted downwardly of the knee of the patient.

In the illustrated embodiment the first and second body mounting portions are first and second brace assemblies. The second brace assembly 204 is pivotably coupled to the first brace assembly 202 via pivot assemblies 206. This makes the orthosis system 200 suited to use in bracing a pivoting joint of the body, such as the knee. Pivoting may be unicentric or polycentric.

In other embodiments the first and second brace assemblies are moveably coupled in some other manner, for example through a sliding coupling. Such embodiments may be suitable for use in bracing an extendable part of the body, for example.

In yet other embodiments, the brace of an orthosis system may be provided as a flexible sleeve, such as a continuous compression sleeve. A first portion of the sleeve is a first body mounting portion to be worn on one side of the user's joint, and a second portion of the sleeve coupled to (i.e. integrally formed with) the first portion is a second body mounting portion to be worn on an opposite side of the user's joint.

In the embodiment of FIGS. 1A and 1B the first brace assembly 202 comprises a frame comprising two frame elements 220. Frame elements 220 are elongate and, in use, are positioned on either side of the portion of the patient's leg immediately above the knee. Frame elements may be formed from a structurally rigid material such as metal, alloy or a rigid plastics material, or a combination of materials. Second brace assembly 204 comprises a frame comprising two frame elements 240. Frame elements 240 are elongate and, in use, are positioned on either side of the portion of the patient's leg immediately below the knee. Frame elements 240 are typically made of the same material as frame elements 220.

Frame elements 220 each comprise a first pivot portion at their lower end (i.e. at an inferior end when the orthosis system 200 is being worn by a patient), and frame elements 240 each comprise a second pivot portion at their upper end (i.e. at a superior end when the orthosis system 200 is being worn by a patient). The first and second pivot portions form part of, and are pivotably coupled at, the pivot assemblies 206.

A sensing module 208 and/or a control module 210 may be provided. The sensing and control modules 208, 210 may be coupled to the pivot assemblies 206 of the body mounting module 102 on either side of the orthosis system 200 and may be removably coupled to the pivot assemblies. For example, when the orthosis system 200 is being worn the sensing module 208 may be provided on one side of the patient's knee and the control module is on the other side of the knee. In some embodiments, a sensor module and/or control module may be provided to the pivot assembly 206 on only one side of the patient's knee, or a sensor and control module may be provided to one or both sides. In other embodiments the orthosis system is configured to mount sensing/control modules in other positions in relation to the body.

In the embodiment of FIGS. 1A and 1B, the body mounting module 102 comprises at least one pad 212 configured to contact the body of the patient (in particular the leg of the patient) in use. The pads 212 may be made of a semi-rigid or soft material to support the orthosis system 200 on the body of the patient in a comfortable manner.

The pads 212 are removably coupled to frame elements 220 or 240 of the body mounting module 102 via pad connectors 214. Pad connectors 214 may enable the orientation and/or position of the pads 212 to be adjusted with respect to the frame elements 220 and 240. This enables the pads to be adjusted to fit different size and shape patients comfortably. Pad connectors 214 may be configured to enable removal and re-attachment of the pads 212 to the frame.

The pad connectors 214 may each comprise a wire that, in use, engages with a ratchet mechanism 216. Ratchet mechanism 216 is configured to adjust the orientation/position of each of the pads 212 relative to the frame elements 220/240 of the body mounting module 102 when the pads 212 are coupled to the frame via the pad connectors 214.

Sensing Module

The sensing module 208 comprises sensor components configured to detect, record, process and/or transmit data relating to the movement and/or rotation of the orthosis system or components thereof. These components may be provided on circuit board.

The sensor components may additionally or alternatively detect, record, process and/or transmit data pertaining to the patient's physical activity and/or physiology. This may include parameters such as joint kinematics (such as joint angle, joint velocity, joint torque, and/or joint acceleration), limb accelerations, limb rotations, limb and/or joint loads, muscle force, muscle strength, muscle velocity, electrical activity, temperature, pH, perspiration, heart rate, blood pressure and/or other bio-signals. Example sensors include rotary encoder, optical and magnetic sensors.

The sensing module 208 may comprise further components to enable the detection and recording of such data. For example, the sensing module 208 may comprise an accelerometer, gyroscope and/or magnetometers. The sensing module 208 may additionally or alternatively comprise physiological sensors, for example a thermometer, electromyography (EMG) sensor, heart rate sensor, blood pressure sensor, blood oxygen level sensor, etc.

Sensing module 208 may comprise a transmitter for transmitting data and/or signals obtained by or through the sensor components to a remote location, for example by RF, Bluetooth, Wi-Fi or any other remote communication protocol. Sensing module 208 may also comprise one or more processors configured to process the data/signals.

The sensor components may further comprise a receiver configured to receive data/signals remotely from an external source, such as external control signals. Data may be stored or received by the sensing module 208 through a physical data storage device such as a memory card, USB stick or the like.

Other sensors may be provided, comprised in or separate from a sensing module 208. For example, the system 100 may also comprise a torque sensing module 104 comprising one or more sensors for monitoring joint interaction torque between the patient and the body mounting module 102. For example, such a sensor(s) may monitor relative displacement between two or more components of the system 100, for example the first and second brace assemblies respectively, this enables a torque sensor to sense torque between the first and second brace assemblies. Torque sensing may be performed when the first and second brace assemblies are locked, or there is some resistance between them. So a torque sensing module may also incorporate a locking mechanism to substantially prevent movement (e.g. rotation) between the first and second brace assemblies, such as described in PCT application PCT/NZ2018/050085.

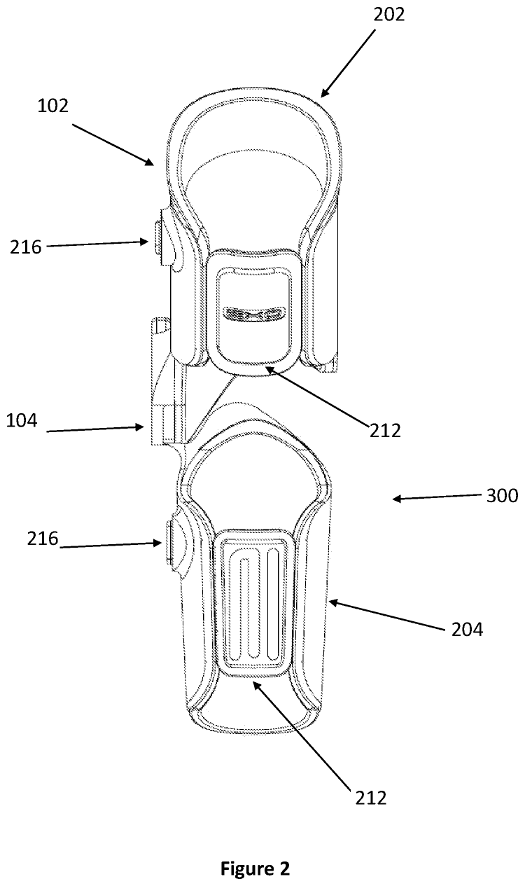

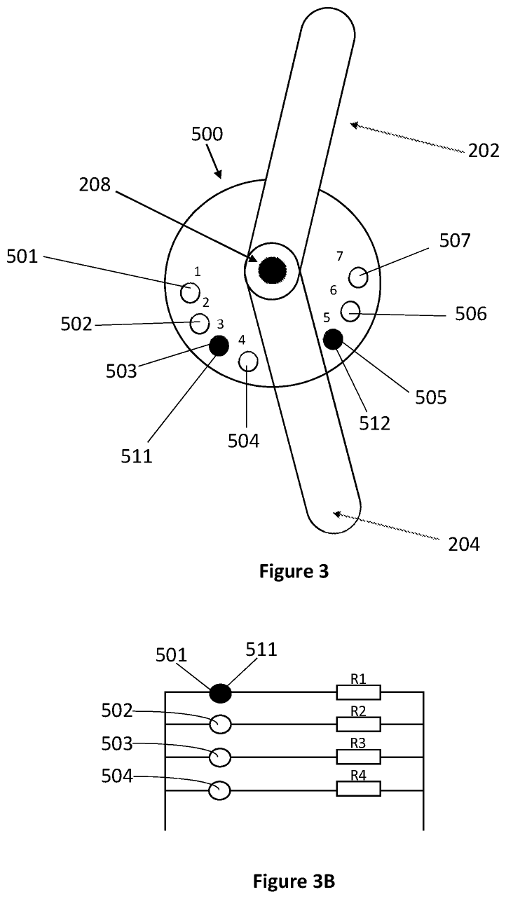

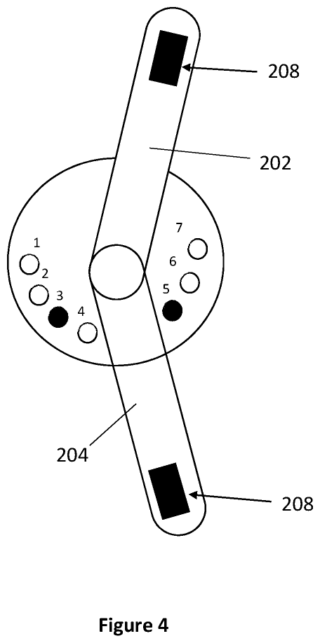

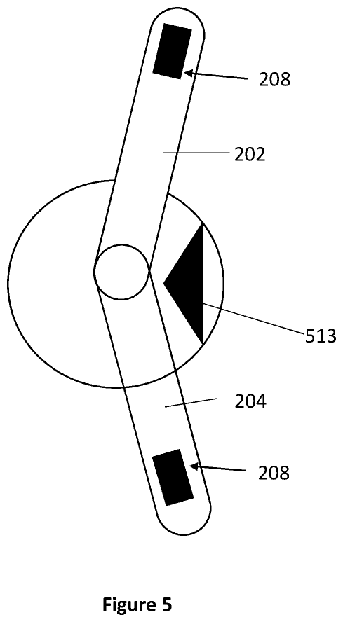

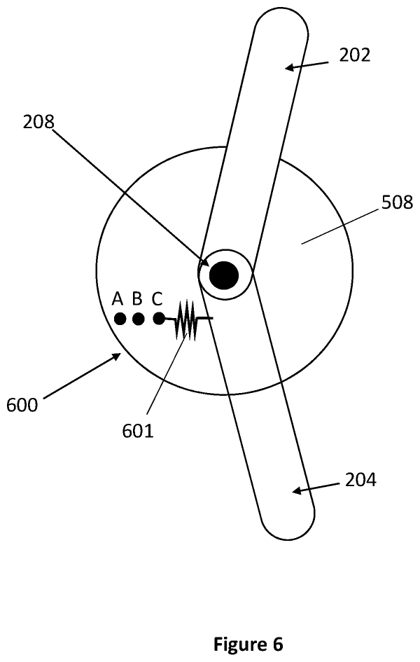

A person skilled in the art will understand that a number of sensor types may be suitable for measuring characteristics of a user's kinematics. For example, a rotary encoder may be used to measure an angle of displacement between the first and second brace assemblies, as shown in schematic representations of FIGS. 3 and 6. Alternatively or additionally an inertial measuring unit(s) (IMU) may be attached to one or each of the first and second brace assemblies to measure the angle of displacement, as indicated schematically in FIGS. 4 and 5. An angle of displacement may be used to infer a resistance to motion level, by calibration of a known resistance element with respect to the amount of relative movement between the brace assemblies, or conversely a resistance measurement such as torque or force may be used to infer angle. A strain gauge may be provided to a compliant/resilient element such as a spring or elastomeric block to measure force or torque, and/or a position of a spring element may be used to indicate a resistance to motion level. A sensor may be positioned remote from the first and/or second brace assembly. For example, an insole sensor may be provided to measure ground reaction force. Where the system including a brace in the form of a flexible sleeve, one or more sensors may be flexible/wearable sensors integrated with the sleeve.

Range of Motion Limitation Mechanism

In preferred embodiments, the orthosis system 100 comprises a configurable range of motion (ROM) limitation system or mechanism 210, to provide a limit on relative displacement, e.g. angular displacement, between the first and second brace assemblies. The ROM limitation mechanism is configurable to adjustably set a ROM between the first and second brace assemblies. The ROM limitation mechanism may allow for a number (plurality) of discrete angle settings, for example, to set extension angle limits or stops of 0°, 10°, 20°, 30° and 40°, and flexion angle limits or stops of 45°, 60°, 75° and 90°. A ROM limitation mechanism (e.g. a rotation limitation module) is described in PCT application PCT/NZ2018/050085.

Example ROM limitation mechanisms are illustrated in schematic form in FIGS. 3 to 5. In FIGS. 3 and 4, the ROM limitation mechanism device 500 comprises a fixed member 508, fixed to one of the first and second brace members 202, 204. The other one of the first and second brace members displaces relative to the fixed member. In the illustrated embodiments, the fixed member 508 is fixed to the first brace member 202, and the second brace member 204 displaces relative to the first brace member and the fixed member. The ROM limitation mechanism comprises at least two ROM stops 511 and 512 to be selectively moved to a plurality of stop or limit positions 501 to 507. For example, each stop 511, 512 may be a pin and each stop position 501 to 507 may comprise an aperture to receive the pin. Each pin can be selectively positioned in any one of the stop positions. Each pin sets a ROM limit by providing a mechanical stop or limit against which the second brace member or component attached to the second brace member contacts. One of the stops 511 determines an extension limit of movement and the other one of the stops 512 determines a flexion limit of movement. In some embodiments, a single limit stop may be provided, for example, to limit movement in the flexion or extension directions.

The ROM limit 511, 512 may be set by an actuator. For example, a linear or rotary actuator may be provided to move a ROM limit 511 or 512 along or around a track between a plurality of positions 501 to 504, 505 to 507.

In FIG. 5, the ROM limitation mechanism comprises an insert 513, to be received on fixed member 508. The system 100 comprises a plurality of inserts of different sizes, each providing a different range of motion with respect to the others. To change the ROM, the user changes out one insert for another from the range of inserts.

Resistance to Motion Module The orthosis system may additionally, or alternatively comprise a resistance to motion system or mechanism, to impart a resistance to motion between the first and second brace assemblies. The RTM mechanism may be comprised in the sensor module 208 or the ROM limitation module 210 or may be provided separately to the system 100.

The resistance mechanism is configurable to provide a range of physical resistance to motion levels. The mechanism may comprise magnetic coupling between the first and second brace assemblies, or configured using mechanical, electrical and/or inductive means. For example, the resistance mechanism may be achieved by the use of one or more compliant/resilient members such as flexible pads or elastomeric members which provides a level of resistance on being twisted or otherwise elastically deformed when the first and second brace assemblies are displaced angularly relative to each other. The resistance to motion mechanism may comprise a spring element to provide a level of resistance to movement between the first and second brace assemblies. The spring element maybe torsional, linear or leaf spring mechanism. The resistance mechanism may also comprise an energy-dissipating mechanism such as a torsional spring or friction brake or may comprise a hydraulic or pneumatic damper. A RTM mechanism is described in PCT application PCT/NZ2018/050085.

An example RTM mechanism is illustrated schematically in FIG. 6. The mechanism 600 comprises a spring element 601. The spring element is coupled between the first and second brace assemblies 202, 204. For example, in the illustrated embodiment the spring element is coupled between the second brace assembly and a member 508 fixed to the first brace assembly. Relative movement between the first and second brace assemblies causes the spring element to compress/extend to provide a resistance to movement. A plurality of resistance levels may be provided by adjusting an amount of compression or tension in the spring element. For example, the amount of compression or tension in a spring may be set by an actuator. In the schematic representation of FIG. 5, the spring element is indicated as a coil or helical spring, in which case a linear actuator may be provided to adjust the resistance level. In an alternative embodiment the spring may be a spiral spring, in which case a rotary actuator may be provided to adjust the resistance level by tightening or loosening the spring to present a desired resistance level.

The ROM limiting mechanism and/or a resistance to motion mechanism may be modular, to be easily interchangeable and with a range of modules provided, each module presenting difference ranges of motion and/or resistance levels. Modular ROM and resistance mechanisms are described in PCT application PCT/NZ2018/050085.

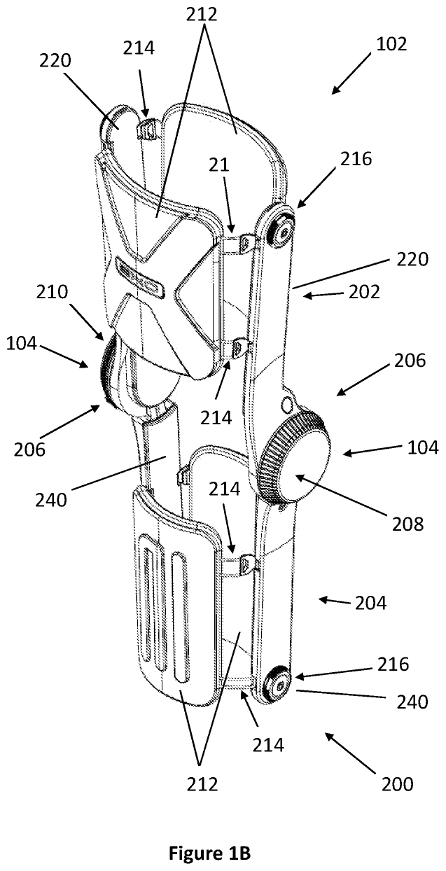

FIG. 2 shows another example orthosis system 300, as described in PCT application PCT/NZ2018/050085. Orthosis system 300 is particularly suited for mounting proximate an elbow (not shown) of the patient (not shown). The system 300 has a body mounting module 102 comprising a first brace assembly 202 and a second brace assembly 204. In use, the first brace assembly 202 is mounted upwardly of the elbow of the patient and the second brace assembly 204 is mounted downwardly of the elbow of the patient. System 300 also has a control/sensor module 104 and may include one or more of the components of system 100 described above.

Orthosis or Exoskeleton System Configuration

According to at least one aspect of the present invention, the orthosis or exoskeleton system 100, 300 is configurable during use based on the user's kinematics (e.g. a user's joint kinematics or gait kinematics) or kinetics (e.g. force required to move a joint against a level of resistance), sensed by one or more sensors 208 of the system.

For example, the system may comprise a plurality of first brace assemblies and a plurality of second brace assemblies. Each of the first and second brace assemblies is of a different size and/or type or configuration with respect to each other. The system is configurable by selectively coupling together a said first and second brace assembly from the plurality of first and second brace assemblies. For example, the system may be configured by changing a rigid brace with a softer and/or less rigid brace. Based on the user's kinematics, the system may communicate to the user, via a user interface of the system or via another device such as a smart phone, that a change in brace assembly or component is required.

In preferred embodiments, the system is configured by updating or setting one or more system settings or set-points/thresholds. For example, the system is configured by setting a ROM limit from a plurality of ROM limits provided by a ROM limitation mechanism, and/or by setting a ROM threshold (e.g. in software), and/or by setting a RTM threshold (e.g. in software). The RTM threshold may correspond with a RTM level from a plurality of RTM levels provided by a RTM mechanism.

As described above, the system may comprise one or more actuators to set a ROM limit and/or RTM level. The system energises one or more actuators to automatically set a ROM limit and/or RTM level based on the user's sensed kinematics. In some embodiments the system automatically sets a ROM threshold and/or RTM threshold, e.g. in software.

For example, where the user achieves a resistance to motion threshold or hits a range of motion limit, or where the user's range of motion is more than a predetermined threshold, the actuator is energised to automatically increase a range of motion limit from a present setting to a new setting to allow for a greater range of motion between the first and second brace assemblies. Alternatively, or in addition to increasing a range of motion limit, the system may increase a resistance to motion level from a present setting to a new higher resistance setting. Once the system has been reconfigured, for example by increasing a physical ROM limit and/or physical RTM level, the system may be further reconfigured by updating one or more setpoints, for example by increasing the ROM threshold and/or the RTM threshold from a present setting to a new (higher) setting. In this way, the system can immediately configure the system based on the user's kinematics to ensure the system is optimally configured to match a user's progress and/or rehabilitation requirements. This can reduce the number of consultations a user may be required to attend during a plan such as a rehabilitation plan.

The system may automatically decrease a resistance level or ROM limit when a user's sensed kinematics indicates the user would be better assisted by a weaker resistance level, or to have a narrower or more limited range of motion. For example, if a range of motion threshold or ROM limit or RTM threshold is/are not achieved in a set time period, the resistance to motion level may be decreased and/or the ROM limit may be decreased from a present limit to a new (lower) limit to present a lower resistance to motion and/or narrower range of motion. Once the limit or level is decreased, the ROM and/or RTM threshold(s) may then be decreased.

The thresholds and/or physical ROM limit/RTM level may be increased or decreased incrementally from a present setting to a next higher or lower setting of a plurality of predetermined thresholds or of a plurality of limits or levels provided by the ROM limitation mechanism or RTM mechanism.

The thresholds (ROM and or RTM) and/or the physical ROM limit and/or RTM level may be changed/updated from a present setting to a new setting until a maximum or minimum setting is reached. If the present setting is less than a maximum, the setting may be increased to a new setting, for example incremented from a present setting to a next (higher) setting. If the present setting is greater than a minimum, the setting may be decreased to a new setting, for example decremented from a present setting to a next (lower) setting.

In some embodiments, the system may be configured by changing a hard limit stop to a soft limit stop, e.g. provided by a spring.

The system may comprise a user interface and/or a communication device, to communicate to the user or to a user device such as a smartphone for example by RF, Bluetooth, Wi-Fi or any other remote communication protocol, that a change to the ROM limit or the RTM level is required. Upon receiving such an indication, the user can make the necessary adjustment to the system, without a requirement to have the user's movement or progress assessed by a medical practitioner or expert.

Other system configurations are possible. For example, the knee brace 100 may be an unloader knee brace, for example for osteoarthritis, wherein adjustment can be made to settings such as an amount of unloading on the medial or lateral side of the joint. The amount of unloading may be updated based on the user's kinematics with an aim to improve gait kinematics and walking speed. Orthosis configuration can be patella support settings.

Determination of System Configuration

As described above, the system is configurable based on a user's kinematics and/or kinetics. The system may comprise a processor to determine a user's kinematics and/or kinetics during use, based on one or more outputs from the sensor(s). Alternatively, or additionally the system may comprise a communication device to communicate sensor data to another user device, for example a smartphone running an application, which determines the user's kinematics/kinetics from the sensor data.

In one embodiment, the system 100 is configured based on the user's ROM being greater than a predetermined threshold, and/or the user achieving a predetermined RTM threshold. For example, where a user's average range of motion (e.g. stroke angle between the first and second brace assemblies) is greater than a ROM threshold, or the user achieves a RTM threshold, the system is configured from a present configuration to an updated configuration, such as adjustment of a physical ROM limit and/or RTM level as described above. The system may be configured from a present configuration to an updated configuration when the user achieves a ROM/RTM threshold more than a predetermined number of times, or more than a predetermined number of times within a predetermined time period. An average may be calculated over a rolling time period.

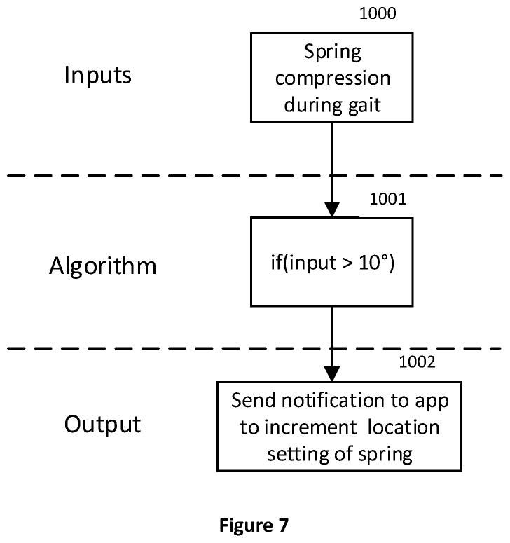

An example condition to configure the system is illustrated by the flow diagram summarised in FIG. 7. Compression of a spring providing a resistance to motion is monitored by one or more sensors to give an indication of a user's gait or ROM (or a level of RTM) (Step 1000). When the user's ROM exceeds a presently set ROM threshold, for example a threshold of 10 degrees (Step 1001), the system sends a notification/signal, either via a user interface/user display, or to an actuator of the system, to increase a setting of the spring to increase the spring resistance and therefore resistance to motion provided by the system to the user. The system may then update/increment the ROM threshold to a new ROM threshold, for example 20 degrees (Step 1002). The system can then continue to monitor the spring compression (Step 1000), and once the user's ROM exceeds the present ROM threshold of 20 degrees (Step 1001), the system sends a further notification or signal, to again increase the spring setting to increment the resistance to motion to be experienced by the user. The ROM threshold may be again updated (from 20 degrees) to a new ROM threshold if a maximum threshold has not been reached (Step 1002).

An indication of the user's kinematics may be provided by the user reaching (i.e. hitting) a ROM limit. In some embodiments, the system is configured from a present configuration to an updated configuration when user reaches a ROM limit, i.e. the user's joint motion achieves a range of motion that hits at least one ROM limit. For example, when the user hits a ROM limit, the system is configured by updating the ROM limit to a new ROM limit and/or updating a RTM level, as described above. The system may be configured from a present configuration to an updated configuration when the user hits a ROM limit more than a predetermined number of times, or more than a predetermined number of times within a predetermined time period.

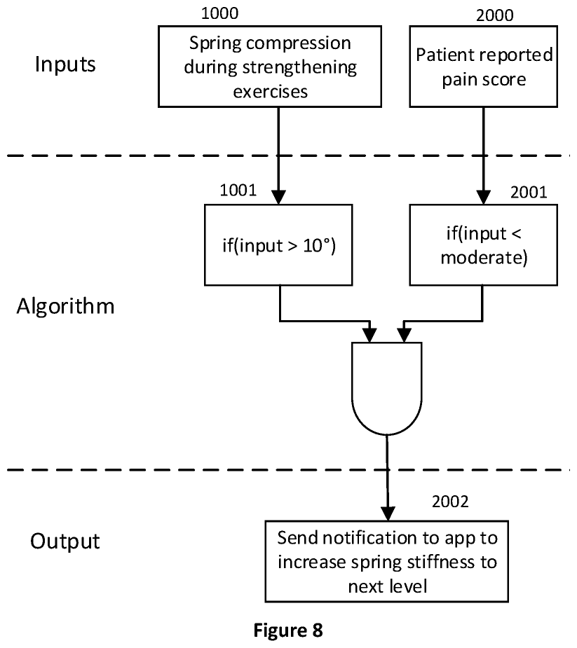

Other measurements of a user's kinematics may be provided. For example, the system may be configured based on a number of steps taken by a user, a user's ground step force (e.g. an average over a rolling time period) and/or a user's pain score input to the system by a user via a user interface or other user device (e.g. smartphone). The method for configuring the system set out in FIG. 8 is the same as in FIG. 7 but with a user's pain score included. The user records a pain score (by entering via a user interface, Step 2000). If the user's pain score is less than a threshold (stated as moderate in this example, Step 2001) and the user's range of motion is greater than a ROM threshold, the system sends a notification/signal, either via a user interface/user display, or to an actuator of the system, to increase a setting of the spring to increase the spring resistance and therefore resistance to motion provided by the system to the user (Step 2002). The system may then update/increment the ROM threshold to a new threshold, for example 20 degrees, as described above with reference to FIG. 7.

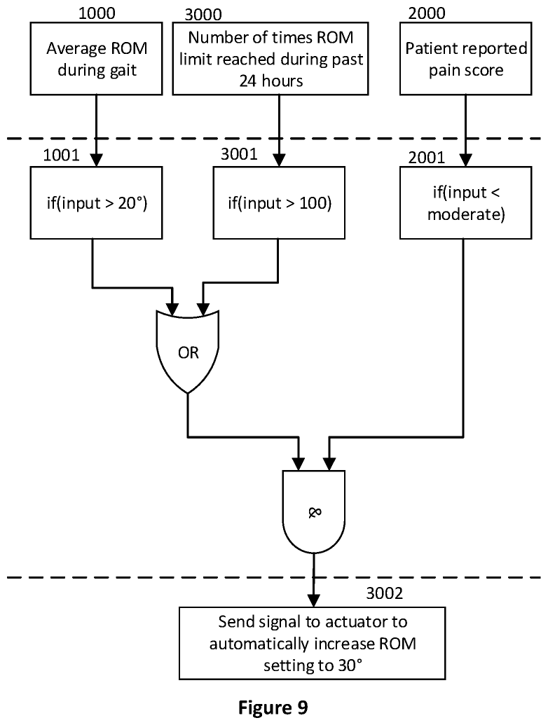

In the example of FIG. 9, each time a user reaches an ROM limit is determined (Step 3000), and if the user hits a ROM limit more than predetermined number of times (100 times) within a time period (previous 24 hours) (Step 3001) or the user has a ROM greater than a threshold (Step 1001, indicated as 20 degrees in FIG. 9), and the user's pain score is less than a moderate level (Step 2001), the system configuration is updated by sending a notification/signal to a user or an actuator to increment a ROM limit setting from a present setting to a new setting (indicated as a ROM limit of 30 degrees, Step 3002). The physical ROM limit may be greater than a corresponding ROM threshold.

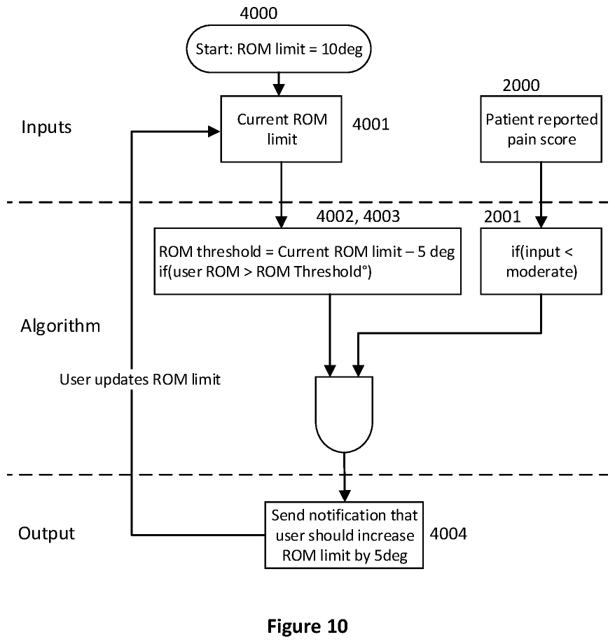

FIG. 10 provides a further example for configuring the system by setting/updating a ROM threshold and ROM limit. An initial ROM limit is set, in the example 10 degrees (Step 4000), which is the current or present ROM limit (Step 4001). The ROM threshold is set as the present ROM limit minus an offset, in this case an offset of 5 degrees (Step 4002). A controller monitors the user's range of motion detected by the one or more sensors, and if the user's range of motion is greater than the present ROM threshold (in this example 5 degrees, Step 4003) the controller causes a change in the ROM limit (Step 4004). The controller may send a signal to the ROM limitation mechanism to update the ROM limit from the present limit to a new limit. Alternatively, the controller provides an indication, such as an audible or visual alarm or indication, to the user, advising the user to update the ROM limit.

Once the ROM limit has been updated, the ROM threshold is updated to the present ROM limit minus the offset (Step 4002). Again, the controller may automatically update the ROM threshold, or may instruct the user to update. In this example, the ROM limit is incremented from a present limit of 10 degrees to a new limit of 15 degrees (Step 4004), and the ROM threshold is increased from 5 degrees to 10 degrees (Step 4002). The control loop of monitoring and updating the ROM limit and the ROM threshold continues until a maximum ROM limit has been reached, at which point the ROM limit is a maximum limit, or alternatively the ROM limit is updated from a maximum limit to no limit, i.e. the ROM limit is removed such that the user can use the system without a physical ROM limit provided. In some embodiments, other inputs may be provided in updating the ROM limit and ROM threshold. In the example of FIG. 10, if the ROM threshold has been reached and the user's pain score is less than moderate then the controller updates/sends notification to the user to update the ROM limit.

The table below provides a further example sequence of updating the ROM limit and ROM threshold during use.

| | ROM | ROM |
|---|---|---|
| Configuration | Limit | Threshold |
| Start | 10 | 7 |
| 2 | 30 | 25 |
| 3 | 60 | 53 |
| 4 | 90 | 80 |
| Finish | No limit | NA |

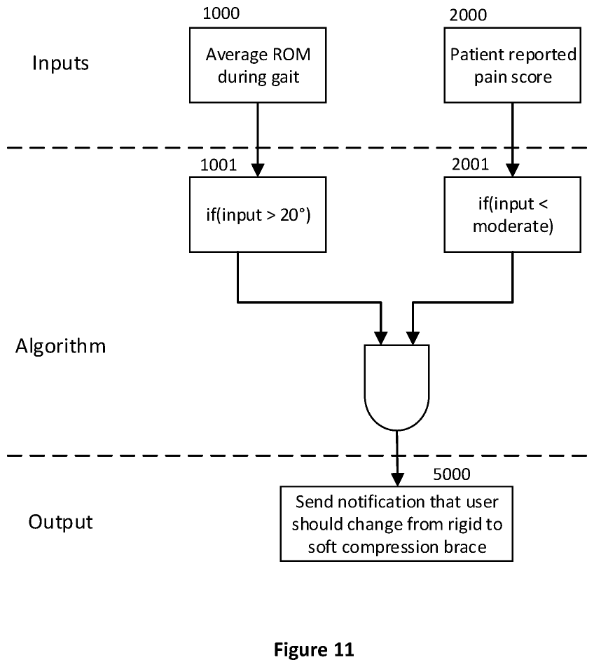

FIG. 11 illustrates an example wherein the system is configured by updating the brace type from a rigid brace comprising rigid first and second body mounting portions coupled together by at least one pivot point to a flexible brace type in the form of a soft compression sleeve brace. When the user's average ROM is greater than a ROM threshold (in this example 20 degrees, Step 1001) and the user's pain score is less than moderate (Step 2001), the system provides an indication to the user to change the brace type (Step 5000).

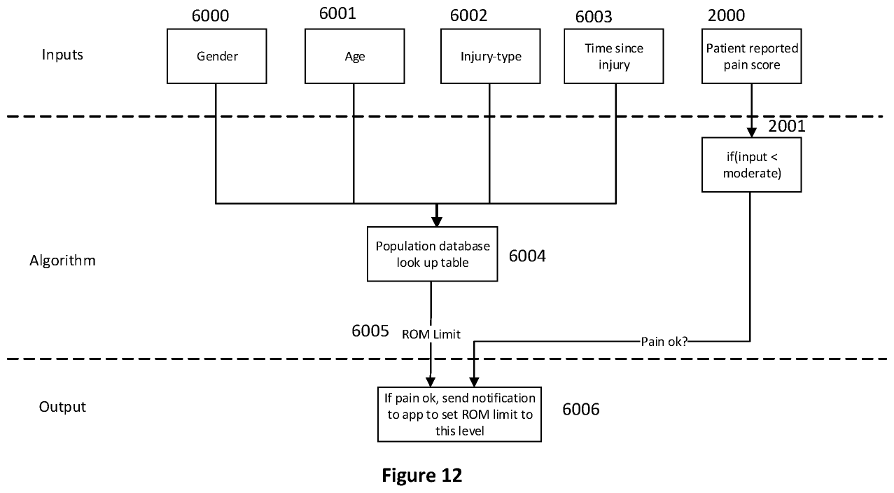

In some embodiments, the system may be configured based on population data. For example, a threshold and or ROM limit or RTM level may be set or updated based on aggregating previous patient recovery data with similar information/demographics, including gender (6000), age (6001), weight, height, injury type (6002) to that of the user, and time since injury (6003). As shown in FIG. 12, population data may be aggregated into an algorithm which may be a look up table or decision tree (6004). A user's data is entered into the algorithm and the algorithm presents a ROM limit (6005), RTM level and/or a ROM/RTM threshold for that user. The system automatically updates or sends a notification to a user to set the limit to the limited determined from the population data (Step 6006). For example, if the user is an 18-year-old male with a grade 1 ACL injury at 2 weeks post operation, the algorithm may indicate a ROM limit should be set to 5 degrees and at 3 weeks post operation the ROM limit should be set to 7 degrees. Further increases/updates may be provided. An operation date may be considered an injury date.

The algorithm/lookup table may be generated by aggregating historical population recovery data, such as which brace ROM limits gave the best patient outcomes determined by the least number of injuries. The algorithm may also consider clinical evidence and clinical knowledge aggregated from many medical practitioners and may take into account other inputs such as a user's reported pain level. In the example of FIG. 12, the ROM limit is set according to the population-based data algorithm (step 6006) if a user's pain score is less than a moderate pain level (Step 2001).

Determining ROM Limit Contact

As described above, in some embodiments, the system is configurable based on the user hitting a ROM limit. The system may comprise an electrical system (circuit) to detect when a limit stop has been hit. An ROM limit being reached is detected by a change in state of the system, for example the electrical system changing from electrically open to electrically closed or vice versa, or a change in resistance, capacitance, inductance. For example, the electrical system may have a first electrical contact associated with the ROM limit and a second electrical contact associated with the first or second brace assembly. The ROM limit being reached is detected when the first and second electrical contacts connect, to complete an electrical circuit. The ROM limit such as a pin as described above with reference to FIG. 3 may be conductive and the first or second brace assembly may have an associated contact, to connect with the conductive ROM limit. Alternatively, the electrical system may include an electrical switch, which is contacted by the first or second brace when a ROM limit has been reached.

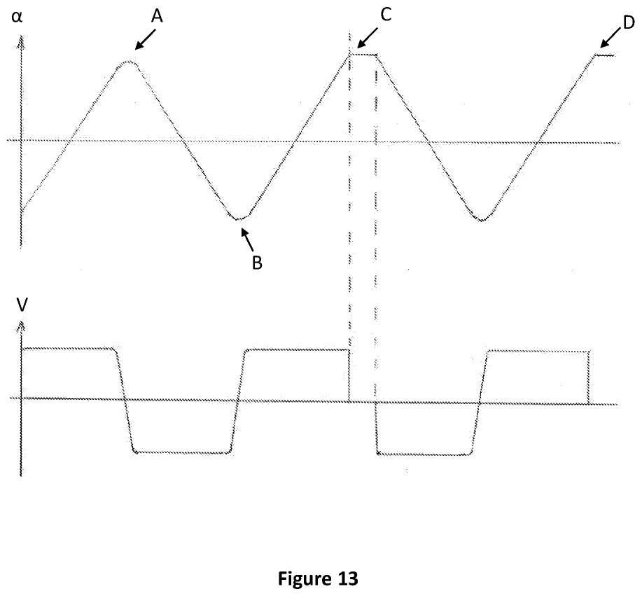

In preferred embodiments, and according to an aspect of the present invention, the ROM limit is determined by the user's kinematics. The ROM limit being reached is determined from one or more derivatives of a relative displacement between the first and the second brace assemblies, e.g. velocity or acceleration of one or both brace assemblies, and/or a rate of change of the resistance to motion between the first and second brace assemblies. FIG. 13 shows a schematic chart of a user's joint angle α (e.g. an angle between the brace assemblies) and the velocity v of the user's limb or relative velocity between brace assemblies (e.g. angular velocity). Where there is no contact with the ROM limit there is a relatively gradual change in motion from extension to flexion or flexion to extension, as indicated at point A and B in FIG. 13. However, when the user hits a limit stop, the change in direction from extension to flexion or flexion to extension is more abrupt, as indicated at points C and D in FIG. 13, and/or an extended period of zero velocity results, as shown at point C. Thus, a contact with the limit stop can be determined by the rate of change in displacement or velocity or possibly higher derivatives of displacement, and/or a change from non-zero velocity to zero velocity, and/or a zero reading for longer than a set time period, and which occurs at a particular angle of displacement between the first and second brace assemblies. That angle gives the angle at which the ROM limit is set.

Reaching the limit stop may be detected by a sensor that monitors displacement, or a velocity sensor or accelerometer, for example, together with processing by a processor, either integrated with the system or a processor of another device such as a user's smartphone. Signal processing may be implemented to filter noise/determine between rapid movements that do not indicate reaching a ROM limit. ROM limits may be determined from a user's kinematics in both the flexion and extension directions of movement.

Once a limit stop has been detected, this information is used as an input to determine how best to configure the system, as described above. Further, where the system allows for a user to input an indication of the ROM limit, the above described embodiments allowing for confirmation of the actual ROM limit position can be used to check the user input is correct, and to issue a warning or alarm if the user input is determined to be incorrect.

A ROM limit may also be determined based on angle/displacement. For example, if there are three ROM limits comprising a minimum, intermediate and maximum limit, if the user's relative displacement was greater than the minimum and intermediate limits, the system can deduce the limit must be configured to the maximum limit. This can be useful in a system that allows a user to enter a limit setting via a user interface. For example, the user may set a mechanical limit stop, and then enter that choice of setting into a processor via a user interface, such that the processor can use the limit setting in methods of configuring the system. The system can then sense check the limit has been entered to the processor correctly. In the above example, the system will determine a user input of minimum or intermediate has been incorrectly entered.

Determining a ROM Limit or RTM Level Setting

In some embodiments and according to another aspect of the present invention, the system has one or more sensors to determine a present ROM limit setting and/or a present RTM level setting, to provide system feedback of an actual/present position of a ROM limit or an actual RTM setting. Again with reference to FIG. 3, the ROM limitation mechanism may comprise one or more limit stops 511 and 512, and a plurality of stop or ROM limit positions 501 to 507. The illustrated system has four extension stops 501 to 504, and three flexion stops 505 to 507. With reference to FIG. 3B, a sensor to determine the position of a ROM limit such as limit 511 may comprise an electrical circuit. The circuit has an electrical path associated with each limit stop position 501 to 504. Each path includes a resistor, R1, R2, R3 or R4, each resistor having a resistance value different to the other resistors. Positioning the limit stop 511 in one of positions 501 to 504 completes the corresponding path associated with the selected position, and the corresponding resistance of the circuit determines the position of the ROM limit 511. Other electrical characteristics may be used to determine the ROM limit setting, for example capacitance or inductance.

A similar circuit may be used to determine a RTM setting. As described above, a RTM may be set by adjusting an amount of compression or tension in a spring element. This may be achieved by selectively moving an end of the spring to one of a plurality of positions, for example positions A, B and C in FIG. 6. A circuit may provide an electrical path associated with each spring position, each with an associated resistor value. Positioning the spring end in one of positions A, B and C completes the corresponding path, and the corresponding resistance of the circuit determines the RTM level.

Unless the context clearly requires otherwise, throughout the description and the claims, the words “comprise”, “comprising”, and the like, are to be construed in an inclusive sense as opposed to an exclusive or exhaustive sense, that is to say, in the sense of “including, but not limited to”.

The entire disclosures of all applications, patents and publications cited above and below, if any, are herein incorporated by reference.

Reference to any prior art in this specification is not, and should not be taken as, an acknowledgement or any form of suggestion that that prior art forms part of the common general knowledge in the field of endeavour in any country in the world.

The invention may also be said broadly to consist in the parts, elements and features referred to or indicated in the specification of the application, individually or collectively, in any or all combinations of two or more of said parts, elements or features.

Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present invention.

1. An orthosis or exoskeleton system to be mounted proximate a user's joint, the system comprising:
a brace comprising a first body mounting portion to be mounted to one side of the user's joint and a second body mounting portion to be mounted to an opposite side of the user's joint, the first and second mounting portions moveably coupled together,
a range of motion (ROM) limitation mechanism presenting a plurality of selectable ROM limits between the first and second body mounting portions,
one or more sensors to detect a user's kinematics and/or kinetics, and
wherein the system is configurable during use based on the user's kinematics and/or kinetics, including by setting or updating the ROM limitation mechanism from a present ROM limit setting to a new ROM limit setting.

2. (canceled)

3. The system as claimed in claim 1, wherein the one or more sensors detect at least a user's range of motion (ROM) between the first and second mounting portions, and
wherein the system is configurable based on the user's ROM being greater than a predetermined ROM threshold, and
the user's ROM being greater than a predetermined ROM threshold is determined by any one or more of:
an average of the user's ROM being greater than a predetermined threshold,
the user's ROM being greater than a predetermined ROM threshold more than a predetermined number of times,
the user's ROM being greater than a predetermined ROM threshold more than a predetermined number of times within a predetermined time period.

4. The system as claimed in claim 3, wherein the predetermined ROM threshold is an angle of displacement between the first and second mounting portions and/or a resistance to motion threshold between the first and second mounting portions.

5-6. (canceled)

7. The system as claimed in claim 1, wherein the sensors detect a ROM limit being reached and/or a resistance to movement (RTM) between the first and second mounting portions, and wherein the system is configurable based on the user reaching an ROM limit and/or a RTM threshold achieved by the user, and
wherein the system is configurable based on any one or more of:
the user hitting a ROM limit and/or achieving a RTM threshold more than a predetermined number of times,
the user hitting a ROM limit and/or achieving a RTM threshold more than a predetermined number of times within a predetermined time period,
an average of the user's RTM being greater than a predetermined threshold.

8-10. (canceled)

11. The system as claimed in claim 1, wherein the system is configurable based on historical data for a population of previous patients with similar information/demographics to the user, the historical data including one or more of gender, age and injury type, and time since injury.

12-15. (canceled)

16. The system as claimed in claim 1, wherein:
the ROM limitation mechanism comprises a plurality of mechanical stops, and wherein the system is configurable by selection of one of the plurality of mechanical stops to set the ROM limit, and/or
wherein the system comprises a resistance to movement (RTM) mechanism presenting a plurality of RTM levels between the first and second mounting portions, and
wherein the RTM mechanism comprises a plurality of resilient members, each providing a RTM level, and wherein the system is configurable by selection of one of the plurality of resilient members to set the RTM level, or
wherein the RTM mechanism comprises a resilient member with a plurality of adjustable levels of tension or compression, and wherein the system is configurable by selection of one of the plurality of levels of tension or compression to set the RTM level.

17. The system as claimed in claim 1, wherein the system comprises a plurality of braces and/or a plurality of first body portions and a plurality of second body mounting portions, each of the braces and/or the first and second mounting portions having different sizes and/or types and/or configurations with respect to each other, and wherein the system is configurable by selecting a said brace and/or selectively coupling together a said first and second mounting portions.

18. The system as claimed in claim 17, wherein the system is configured by updating the brace from a rigid or semi-rigid brace to a compression sleeve brace.

19-20. (canceled)

21. The system as claimed in claim 1, wherein the system is configurable during use by setting or updating an ROM threshold and/or a RTM threshold from a present threshold to a new threshold, and wherein the new ROM threshold is equal to the new ROM limit minus an offset.

22. (canceled)

23. The system as claimed in claim 1, wherein the sensors detect a ROM limit being reached and/or a resistance to movement (RTM) between the first and second mounting portions, and wherein the system is configurable based on the user reaching an ROM limit and/or a RTM threshold achieved by the user, and
wherein the system determines a ROM limit has been reached based on the user's kinematics and/or kinetics by determining:
a ROM limit has been reached based on one or more derivatives of a relative displacement (position) and/or a rate of change of RTM between the first and second mounting portions, or
a ROM limit has been reached based on a rate of change in relative displacement and/or velocity between the first and second mounting portions.

24-25. (canceled)

26. The system as claimed in claim 23, wherein the system determines a ROM limit has been reached based on a change in velocity between the first and second mounting portions from non-zero velocity to zero velocity, and/or when the velocity between the first and second mounting portions is zero for a predetermined time period, and wherein the ROM limit is defined by an angle of displacement between the first and second body mounting portions when the velocity between the first and second mounting portions is zero or zero for the predetermined time period.

27-28. (canceled)

29. A method for configuring an orthosis or exoskeleton system comprising a brace, the brace having a first body mounting portion to be mounted to one side of the user's joint and a second body mounting portion to be mounted to an opposite side of the user's joint, the first and second mounting portions moveably coupled together, and a range of motion (ROM) limitation mechanism presenting a plurality of selectable ROM limits between the first and second body mounting portions, the method comprising:
measuring a user's kinematics and/or kinetics, and
configuring the system during use based on the user's kinematics and/or kinetics, including configuring the system by setting or updating the ROM limitation mechanism from a present ROM limit setting to a new ROM limit setting.

30. (canceled)

31. The method as claimed in claim 29, wherein the method comprises:
measuring at least a range of motion (ROM) between the first and second brace, and
configuring the system based on the user's ROM being greater than a predetermined ROM threshold, including determining the user's ROM is greater than a predetermined ROM threshold by any one or more of:
an average of the user's ROM being greater than a predetermined threshold,
the user's ROM being greater than a predetermined ROM threshold more than a predetermined number of times,
the user's ROM being greater than a predetermined ROM threshold more than a predetermined number of times within a predetermined time period.

32-33. (canceled)

34. The method as claimed in claim 29, wherein the method comprises detecting a ROM limit being reached and/or a resistance to movement (RTM) between the first and second body mounting portions, and configuring the system based on the user reaching an ROM limit and/or the user reaching the user reaching a RTM threshold, and
wherein the method comprises configuring the system based on any one or more of:
the user hitting a ROM limit and/or achieving a RTM threshold more than a predetermined number of times,
the user hitting a ROM limit and/or achieving a RTM threshold more than a predetermined number of times within a predetermined time period,
an average of the user's RTM being greater than a predetermined threshold.

35-36. (canceled)

37. The method as claimed in claim 29, wherein the method comprises configuring the system based on historical data for a population of previous patients with similar information/demographics to the user, the historical data including one or more of gender, age and injury type, and time since injury.

38-41. (canceled)

42. The method as claimed in claim 29, wherein the system comprises a plurality of braces and/or a plurality of first body mounting portions and a plurality of second mounting portions, each of the braces and/or the first and second body mounting portions having different sizes and/or configurations with respect to each other, and wherein the method comprises indicating to the user that a change in the brace and/or the first and/or second body mounting portions is required.

43. The method as claimed in claim 42, wherein the method comprises instructing the user to update the brace from a rigid or semi-rigid brace to a compression sleeve brace.

44-45. (canceled)

46. The method as claimed in claim 29, wherein the method comprises configuring the system during use by setting or updating an ROM threshold and/or a RTM threshold from a present threshold to a new threshold, and wherein the new ROM threshold is equal to the new ROM limit minus a ROM offset.

47. (canceled)

48. The method as claimed in claim 29, wherein the method comprises incrementing or decrementing a ROM limit or RTM level from a present limit or level to a new limit or level if the present limit or level is less than a maximum or greater than a minimum limit or level, and
determining an ROM limit has been reached based on the user's kinematics by determining:
a ROM limit has been reached based on one or more derivatives of a relative displacement (position) and/or a rate of change of RTM between the first and second mounting portions, or
determining a ROM limit has been reached based on a rate of change in relative displacement and/or velocity between the first and second mounting portions.

49-50. (canceled)

51. The method as claimed in claim 48, wherein the system determines a ROM limit has been reached based on:
a change in velocity between the first and second mounting portions from non-zero velocity to zero velocity, and/or
when the velocity between the first and second mounting portions is zero for a predetermined time period, and wherein the ROM limit is defined by an angle of displacement between the first and second body mounting portions when the velocity between the first and second mounting portions is zero or zero for the predetermined time period.
